(19) United States
(12) Patent Application Publication
(10) Pub. No.: US 2019/0099304 A1
(43) Pub. Date: Apr. 4, 2019

(54) Personal Hygiene Device

(71) Applicant: Carol Berry, Cape Coral, FL (US)

(72) Inventor: Carol Berry, Cape Coral, FL (US)

(21) Appl. No.: 15/724,819

(22) Filed: Oct. 4, 2017

(51) Int. Cl. A61F 13/491; A61F 13/49; A61F 13/471; A61F 13/62

(52) U.S. Cl. CPC A61F 13/4915; A61F 13/49009; A61F 2013/49092; A61F 13/62; A61F 2013/49087; A61F 13/471

(57) ABSTRACT

A personal hygiene device for nighttime diapering of an adult male includes a shell that has a top, which is open. The shell is shaped substantially complementarily to a lower trunk of an adult male and is water-impermeable. A pair of openings is positioned in the shell to insert legs of a user to position the shell over the lower trunk of the user. A first pad is coupled to an interior surface of the shell. The first pad is configured to absorb urine that flows from a penis of the user. A panel is positioned over a bottom section of the first pad. The panel has a bottom edge and opposing side edges that are coupled to the shell to define a sleeve. The sleeve is configured to insert the penis of the user to position the penis adjacent to the first pad.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to personal hygiene devices and more particularly pertains to a new personal hygiene device for nighttime diapering of an adult male.

### BRIEF SUMMARY OF THE INVENTION

An embodiment of the disclosure meets the needs presented above by generally comprising a shell that has a top, which is open. The shell is shaped substantially complementarily to a lower trunk of an adult male and is water-impermeable. A pair of openings is positioned in the shell to insert legs of a user to position the shell over the lower trunk of the user. A first pad is coupled to an interior surface of the shell. The first pad is configured to absorb urine that flows from a penis of the user. A panel is positioned over a bottom section of the first pad. The panel has a bottom edge and opposing side edges that are coupled to the shell to define a sleeve. The sleeve is configured to insert the penis of the user to position the penis adjacent to the first pad.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a personal hygiene device according to an embodiment of the disclosure.

FIG. 2 is a side view of an embodiment of the disclosure.

FIG. 3 is a top view of an embodiment of the disclosure.

FIG. 4 is a cross-sectional view of an embodiment of the disclosure.

### DETAILED DESCRIPTION OF THE INVENTION

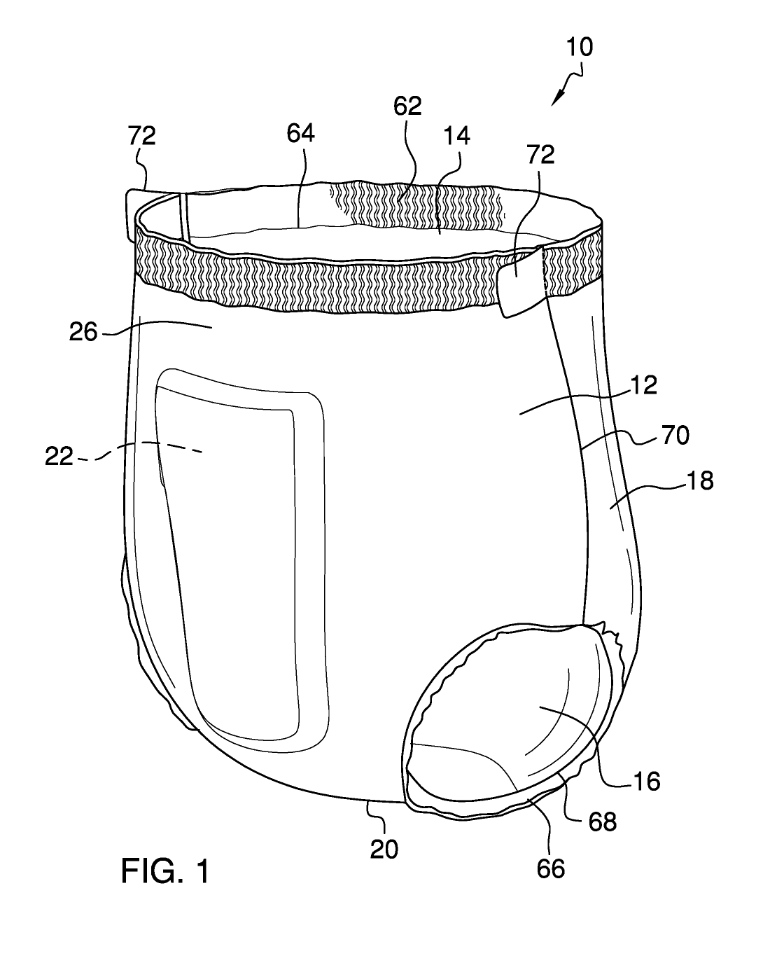

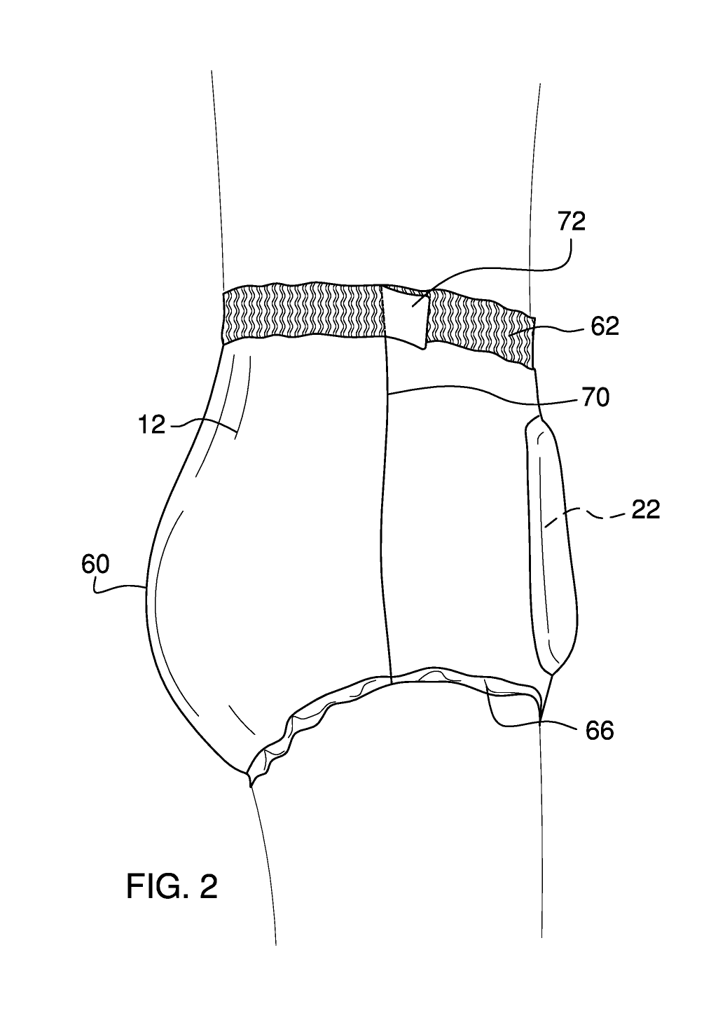

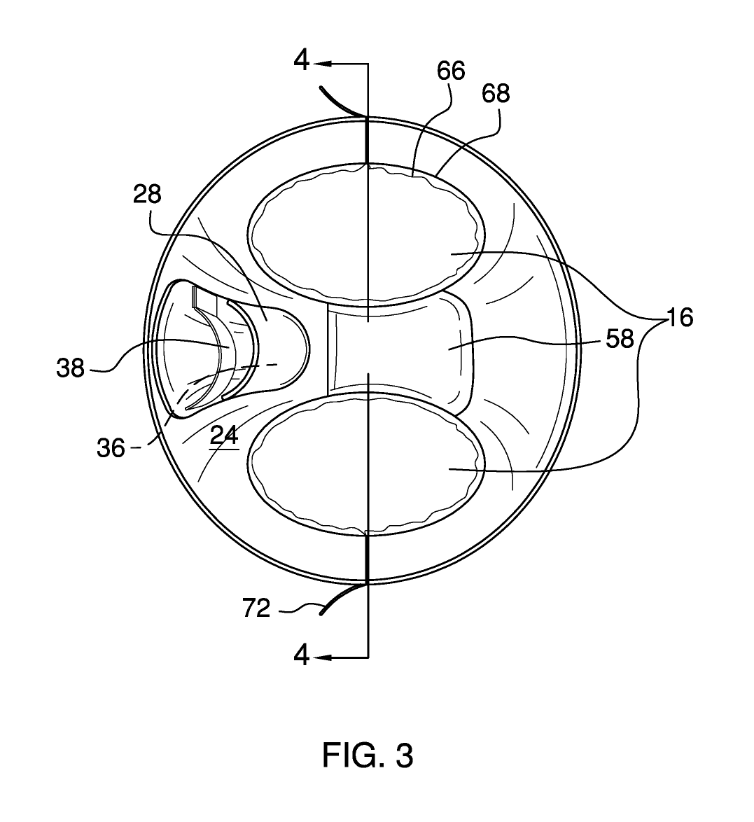

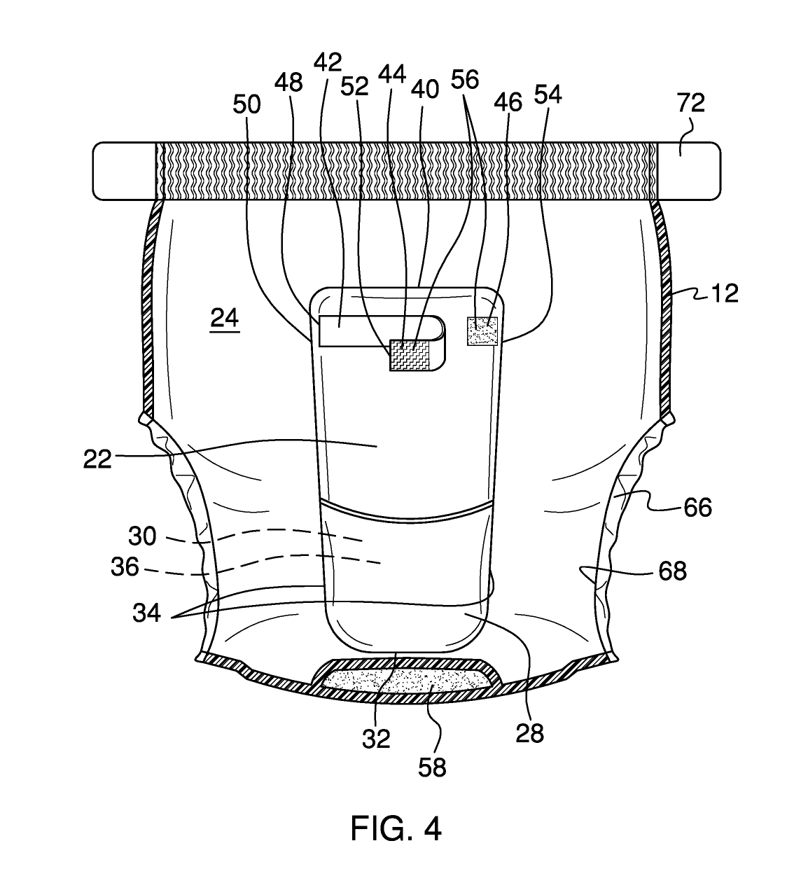

With reference now to the drawings, and in particular to FIGS. 1 through 4 thereof, a new personal hygiene device embodying the principles and concepts of an embodiment of the disclosure and generally designated by the reference numeral 10 will be described.

As best illustrated in FIGS. 1 through 4, the personal hygiene device 10 generally comprises a shell 12 that has a top 14. The top 14 is open. The shell 12 is shaped substantially complementarily to a lower trunk of an adult male. The shell 12 is water-impermeable.

Each of a pair of openings 16 is positioned in a respective opposing side 18 of the shell 12 proximate to a bottom 20 of the shell 12. Each opening 16 is configured to insert a respective leg of a user to position the shell 12 over the lower trunk of the user.

A first pad 22 is coupled to an interior surface 24 of the shell 12. The first pad 22 is configured to absorb urine. The first pad 22 is positioned on a front 26 of the shell 12 equally distant from the openings 16. The first pad 22 is configured to absorb the urine that flows from a penis of the user. In one embodiment, the first pad 22 is substantially rectangularly shaped.

A panel 28 is positioned over a bottom section 30 of the first pad 22. The panel 28 has a bottom edge 32 and opposing side edges 34 that are coupled to the shell 12 to define a sleeve 36. The sleeve 36 is configured to insert the penis of the user to position the penis adjacent to the first pad 22.

A coupler 38 is coupled to the first pad 22 proximate to an upper end 40 of the first pad 22. The coupler 38 is configured to selectively couple to the penis of the user to retain the penis in the sleeve 36. In one embodiment, the coupler 38 comprises a strap 42, a first connector 44, and a second connector 46. The strap 42 is coupled by a first end 48 to the first pad 22 proximate to a first side 50 of the first pad 22. The first connector 44 is coupled to the strap 42 proximate to a second end 52 of the strap 42. The second connector 46 is coupled to the first pad 22 proximate to a second side 54 of the first pad 22. The strap 42 is configured to loopedly position around the penis of the user to position the first connector 44 to couple to the second connector 46. The penis of the user is coupled to the first pad 22 and is retained in the sleeve 36. In one embodiment, the first connector 44 and the second connector 46 comprises a hook and loop fastener 56.

In one embodiment, a second pad 58 is coupled to the interior surface 24 of the shell 12. The second pad 58 is configured to absorb liquids. The second pad 58 is positioned on the bottom 20 of the shell 12 between the openings 16. The second pad 58 extends toward a back 60 of the shell 12. The second pad 58 is configured to absorb the liquids that are excreted from the user into the shell 12.

In another embodiment, a first band 62 is coupled to a perimeter 64 of the top 14. The first band 62 is elastomeric so that the perimeter 64 of the top 14 is sealably couplable to the lower trunk of the user. The first band 62 is configured to sealably couple the perimeter 64 to the waist of the user to prevent leakage of the urine from the top 14 of the shell 12.

In yet another embodiment, each of a pair of second bands 66 is coupled to a circumference 68 of a respective opening 16. The second bands 66 are elastomeric so that the circumference 68 of the opening 16 is sealably couplable to the respective leg of the user. Each second band 66 is configured to sealably couple an associated circumference 68 to the respective leg of the user to prevent leakage of the urine from the respective opening 16.

In still yet another embodiment, each of a pair of partial perforation lines 70 is positioned in a respective opposing side 18 of the shell 12. Each partial perforation line 70 extends from the top 14 to a respective opening 16. Each partial perforation line 70 is configured to be torn to facilitate removal of the shell 12 from the lower trunk of the user.

In still yet another embodiment, a pair of tabs 72 is coupled to and extends from the shell 12. Each tab 72 is positioned proximate to the top 14 and an associated partial perforation line 70. Each tab 72 is configured to be grasped in digits of a hand of the user and to be pulled to tear the shell 12 along the associated partial perforation line 70 to facilitate the removal of the shell 12 from the lower trunk of the user.

In use, each opening 16 is configured to insert the respective leg of the user to position the shell 12 over the lower trunk of the user. The sleeve 36 is configured to insert the penis of the user to position the penis adjacent to the first pad 22. The strap 42 is configured to loopedly position around the penis of the user to position the first connector 44 to couple to the second connector 46. The penis of the user is coupled to the first pad 22 and is retained in the sleeve 36. The first pad 22 is configured to absorb the urine that flows from the penis of the user. The second pad 58 is configured to absorb the liquids that are excreted from the user into the shell 12. The first band 62 is configured to sealably couple the perimeter 64 to the waist of the user to prevent leakage of the urine from the top 14 of the shell 12. Each second band 66 is configured to sealably couple an associated circumference 68 to the respective leg of the user to prevent leakage of the urine from the respective opening 16. Each tab 72 is configured to be grasped in the digits of the hand of the user and to be pulled to tear the shell 12 along the associated partial perforation line 70 to facilitate the removal of the shell 12 from the lower trunk of the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A personal hygiene device comprising:
a shell having a top, said top being open, said shell being shaped substantially complementarily to a lower trunk of an adult male, said shell being water-impermeable;
a pair of openings, each said opening being positioned in a respective opposing side of said shell proximate to a bottom of said shell;
a first pad coupled to an interior surface of said shell, said first pad being configured for absorbing urine, said first pad being positioned on a front of said shell equally distant from said openings;
a panel positioned over a bottom section of said first pad, said panel having a bottom edge and opposing side edges coupled to said shell defining a sleeve; and
wherein said openings are positioned in said shell such that each said opening is configured for inserting a respective leg of a user to position said shell over the lower trunk of the user, wherein said first pad is positioned on said shell such that said first pad is configured for absorbing the urine flowing from a penis of the user, wherein said panel is positioned on said shell such that said sleeve is configured for inserting the penis of the user for positioning the penis adjacent to said first pad.

2. The device of claim 1, further including said first pad being substantially rectangularly shaped.

3. The device of claim 1, further including a coupler coupled to said first pad proximate to an upper end of said first pad, said coupler being configured for selectively coupling to the penis of the user, wherein said coupler is positioned on said first pad such that said coupler is configured for coupling to the penis of the user for retaining the penis is said sleeve.

4. The device of claim 3, further including said coupler comprising a strap, a first connector, and a second connector, said strap being coupled by a first end to said first pad proximate to a first side of said first pad, said first connector being coupled to said strap proximate to a second end of said strap, said second connector being coupled to said first pad proximate to a second side of said first pad, wherein said strap is positioned on said first pad such that said strap is configured for loopedly positioning around the penis of the user positioning said first connector for coupling to said second connector to couple the penis of the user to said first pad such that the penis of the user is retained in said sleeve.

5. The device of claim 4, further including said first connector and said second connector comprising a hook and loop fastener.

6. The device of claim 1, further including a second pad coupled to said interior surface of said shell, said second pad being configured for absorbing liquids, said second pad being positioned on said bottom of said shell between said openings, said second pad extending toward a back of said shell, wherein said second pad is positioned on said shell such that said second pad is configured for absorbing the liquids excreted from the user into said shell.

7. The device of claim 1, further including a first band coupled to a perimeter of said top, said first band being elastomeric such that said perimeter of said top is sealably couplable to the lower trunk of the user, wherein said first band is positioned on said shell such that said first band is configured for sealably coupling said perimeter to the waist of the user for preventing leaking of the urine from said top of said shell.

8. The device of claim 1, further including a pair of second bands, said second bands being elastomeric, each said second band being coupled to a circumference of a respective said opening such that said circumference of said opening is sealably couplable to the respective leg of the user, wherein said second bands are positioned on said shell such that each said second band is configured for sealably coupling an associated said circumference to the respective leg of the user for preventing leaking of the urine from said respective said opening.

9. The device of claim 1, further including a pair of partial perforation lines, each said partial perforation line being positioned in a respective said opposing side of said shell, each said partial perforation line extending from said top to a respective said opening, wherein said pair of partial perforation lines is positioned in said shell such that each said partial perforation line is configured for tearing for facilitating removing of said shell from the lower trunk of the user

10. The device of claim 9, further including a pair of tabs coupled to and extending from said shell, each said tab being positioned proximate to said top and an associated said partial perforation line, wherein said tabs are positioned on said shell such that each said tab is configured for grasping in digits of a hand of the user and for pulling for tearing of said shell along said associated said partial perforation line for facilitating removing of said shell from the lower trunk of the user.

11. A personal hygiene device comprising:
a shell having a top, said top being open, said shell being shaped substantially complementarily to a lower trunk of an adult male, said shell being water-impermeable;
a pair of openings, each said opening being positioned in a respective opposing side of said shell proximate to a bottom of said shell, wherein said openings are positioned in said shell such that each said opening is configured for inserting a respective leg of a user to position said shell over the lower trunk of the user;
a first pad coupled to an interior surface of said shell, said first pad being configured for absorbing urine, said first pad being positioned on a front of said shell equally distant from said openings, wherein said first pad is positioned on said shell such that said first pad is configured for absorbing the urine flowing from a penis of the user, said first pad being substantially rectangularly shaped;
a panel positioned over a bottom section of said first pad, said panel having a bottom edge and opposing side edges coupled to said shell defining a sleeve, wherein said panel is positioned on said shell such that said sleeve is configured for inserting the penis of the user for positioning the penis adjacent to said first pad;
a coupler coupled to said first pad proximate to an upper end of said first pad, said coupler being configured for selectively coupling to the penis of the user, wherein said coupler is positioned on said first pad such that said coupler is configured for coupling to the penis of the user for retaining the penis is said sleeve, said coupler comprising a strap, a first connector, and a second connector, said strap being coupled by a first end to said first pad proximate to a first side of said first pad, said first connector being coupled to said strap proximate to a second end of said strap, said second connector being coupled to said first pad proximate to a second side of said first pad, wherein said strap is positioned on said first pad such that said strap is configured for loopedly positioning around the penis of the user positioning said first connector for coupling to said second connector to couple the penis of the user to said first pad such that the penis of the user is retained in said sleeve, said first connector and said second connector comprising a hook and loop fastener;
a second pad coupled to said interior surface of said shell, said second pad being configured for absorbing liquids, said second pad being positioned on said bottom of said shell between said openings, said second pad extending toward a back of said shell, wherein said second pad is positioned on said shell such that said second pad is configured for absorbing the liquids excreted from the user into said shell;
a first band coupled to a perimeter of said top, said first band being elastomeric such that said perimeter of said top is sealably couplable to the lower trunk of the user, wherein said first band is positioned on said shell such that said first band is configured for sealably coupling said perimeter to the waist of the user for preventing leaking of the urine from said top of said shell;
a pair of second bands, said second bands being elastomeric, each said second band being coupled to a circumference of a respective said opening such that said circumference of said opening is sealably couplable to the respective leg of the user, wherein said second bands are positioned on said shell such that each said second band is configured for sealably coupling an associated said circumference to the respective leg of the user for preventing leaking of the urine from said respective said opening;
a pair of partial perforation lines, each said partial perforation line being positioned in a respective said opposing side of said shell, each said partial perforation line extending from said top to a respective said opening, wherein said pair of partial perforation lines is positioned in said shell such that each said partial perforation line is configured for tearing for facilitating removing of said shell from the lower trunk of the user;
a pair of tabs coupled to and extending from said shell, each said tab being positioned proximate to said top and an associated said partial perforation line, wherein said tabs are positioned on said shell such that each said tab is configured for grasping in digits of a hand of the user and for pulling for tearing of said shell along said associated said partial perforation line for facilitating removing of said shell from the lower trunk of the user; and
wherein said openings are positioned in said shell such that each said opening is configured for inserting the respective leg of the user to position said shell over the lower trunk of the user, wherein said panel is positioned on said shell such that said sleeve is configured for inserting the penis of the user for positioning the penis adjacent to said first pad, wherein said strap is positioned on said first pad such that said strap is configured for loopedly positioning around the penis of the user positioning said first connector for coupling to said second connector to couple the penis of the user to said first pad such that the penis of the user is retained in said sleeve, wherein said first pad is positioned on said shell such that said first pad is configured for absorbing the urine flowing from the penis of the user, wherein said second pad is positioned on said shell such that said second pad is configured for absorbing the liquids excreted from the user into said shell, wherein said first band is positioned on said shell such that said first band is configured for sealably coupling said perimeter to the waist of the user for preventing leaking of the urine from said top of said shell, wherein said second bands are positioned on said shell such that each said second band is configured for sealably coupling an associated said circumference to the respective leg of the user for preventing leaking of the urine from said respective said opening, wherein said tabs are positioned on said shell such that each said tab is configured for grasping in digits of a hand of the user and for pulling for tearing of said shell along said associated said partial perforation line for facilitating removing of said shell from the lower trunk of the user.
